(19) United States
(12) Patent Application Publication
(10) Pub. No.: US 2005/0085545 A1
(43) Pub. Date: Apr. 21, 2005

(54) Cycloalkylaminoacid compounds, processes for making and uses thereof

(75) Inventors: Carl Alan Busacca, Poughkeepsie, NY (US); Karl Georg Grozinger, Ridgefield, CT (US)

Correspondence Address: MICHAEL P. MORRIS;BOEHRINGER INGELHEIM CORPORATION, 900 RIDGEBURY ROAD, P O BOX 368, RIDGEFIELD, CT, 06877-0368, US

(73) Assignee: Boehringer Ingelheim International GmbH, Ingelheim (DE)

(21) Appl. No.: 10/925,327

(22) Filed: Aug. 24, 2004

Related U.S. Application Data
(60) Provisional application No. 60/498,559, filed on Aug. 27, 2003.

(51) Int. Cl. A61K 31/195; C07C 229/48

(52) U.S. Cl. 514/561; 514/562; 562/507

(57) ABSTRACT

The invention relates to the field of pharmaceutics and more specifically to compositions useful in the preparation of cycloalkyaminoacids and processes for making cycloaminoacids 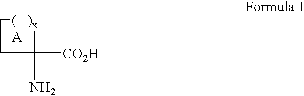
wherein A is a cycloalkyl optionally partially or fully halogenated and optionally substituted with one or more OH, NH₂, SO₂, phenyl or CF₃;
X is C₀₋₈.

### APPLICATION DATA

This application claims benefit to U.S. provisional application No. 60/498,559 filed Aug. 27, 2004 and is incorporated herein by reference.

### FIELD OF THE INVENTION

The invention relates to the field of pharmaceutics and more specifically to compositions useful in the preparation of cycloalkyaminoacids and processes for making cycloalkylaminoacids.

### BACKGROUND OF THE INVENTION

Cycloalkylaminoacids are useful compounds in the preparation of pharmaceutical agents. For instance, Cyclobutaneaminoacids are useful in peptide synthesis and for use in Boron neutron capture therapy (BNCT) for cancer treatment (Refs. Kabalka, G. W.; Yao, M.-L., Tetrahedron Lett., 2003, 1879-1881. Srivastava, R. R.; Singhaus, R. R. and Kabalka, G. W. J Org. Chem. 1999, 64, 8495-8500. Srivastava, R. R.; Kabalka, G. W. J Org. Chem. 1997, 62, 8730-8734. Srivastava, R. R.; Singhaus, R. R. and Kabalka, G. W. J. Org. Chem. 1997, 62, 4476-4478.). Consequently, there is a need in the art for a scaleable synthetic route for making these products using materials that are inexpensive and easy to work with.

There are few reported routes for the synthesis of cycloalkylamino acids in the art. In 1937 Demyanov reported a preparation of the compound shown in Scheme I from cyclobutanediamide by rearrangement to the hydantoin followed by basic hydrolysis.

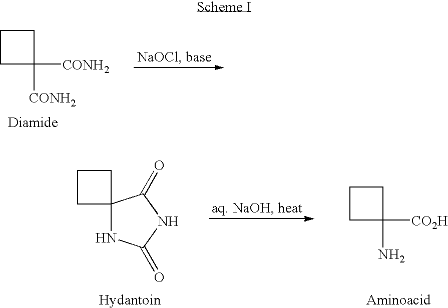

(Demyanov, N. A.; Tel'nov, S. M. Izv. Akad. Nauk. SSSR, Ser. Khim. 1937, 529), and described again in 1964 (Dvonch, W.; Fletcher, H.; Album, H. E. J. Org. Chem. 1964, 29, 2764). Modern variations of this scheme for different targets can be found in: Tanaka, K.-I.; Iwabuchi, H.; Sawanishi, H. Tetrahedron: Asymmetry 1995, 6(9), 2271.

The Strecker reaction is also a known method for the preparation of aminoacids from ketones and aldehydes. Strecker, A. Ann. 1850, 75, 27; For a review see: Barrett, G. C., Chemistry and Biochemistry of the Aminoacids (Chapman and Hall, New York, 1985), pp 251-261. Strecker reaction have also been used on oxetanones. Kozikowski, A. P.; Fauq, A. H. Synlett 1991, 783.

Conversion of cyclobutanone to hydantoin has been reported. Goodman, M.; Tsang, J. W.; Schmied, B.; Nyfeler, R. J. Med. Chem. 1984, 27, 1663. Coomeyras, A.; Rousset, A.; Lasperas, M. Tetrahedron 1980, 36, 2649.

Another route for making cycloalkylaminoacids is through a Curtis rearrangement as shown in Scheme II below. Haefliger, W.; Kloppner, E. Helv. Chim. Acta 1982, 65, 1837).

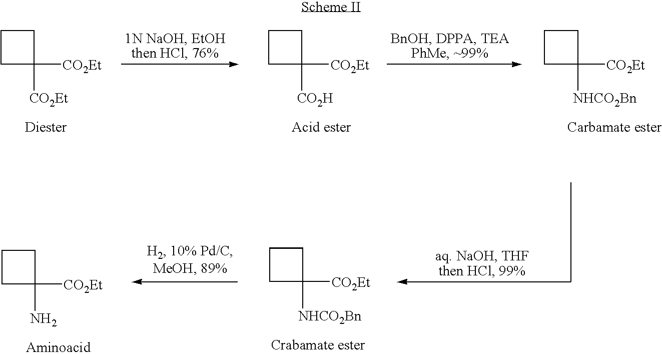

Hofmann rearrangements of acid amides have also been reported. Huang, Lin and Li, J. Chin. Chem. Soc., 1947, 15, 33-50; Lin, Li and Huang, Sci. Technol. China, 1948, 1, 9; Huang, J. Chin. Chem. Soc., 1948, 15, 227: M. L., Izquierdo, I. Arenal, M. Bernabe, E. Alvearez, E. F., Tetrahedron, 1985, 41, 215-220: Zitsane, D. R.; Ravinya, I. T.; Riikure, I. A.; Tetere, Z. F.; Gudrinietse, E. Yu.; Kalei, U. O.; Russ. J. Org. Chem.; EN; 35; 10; 1999; 1457-1460; Zorkae; Zh. Org. Khim.; RU; 35; 10; 1999; 1489-1492. For Hofmann reaction using NBS/DBU have also been described: X. Huang, M. Seid, J. W, Keillor, J. Org. Chem. 1997, 62, 7495-7496.

### DESCRIPTION OF THE INVENTION

The broadest aspect of the invention provides for cycloalkylaminoacid compounds of Formula I: 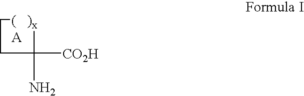
wherein
A is a cycloalkyl optionally partially or fully halogenated and optionally substituted with one or more OH, NH₂, C₁₋₆, SO₂, phenyl or CF₃;
X is C₀₋₈
   and pharmaceutically acceptable salts, salts, solvates, hydrates, stereoisomers, optical isomers; enatiomers, diastereoisomes and racemeic mixtures, esters, tautomers, individual isomers, and mixtures of isomers thereof.

The invention also relates to processes for preparing cycloalkylaminoacids of Formula I 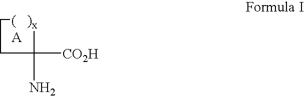
and is comprised of the steps of: 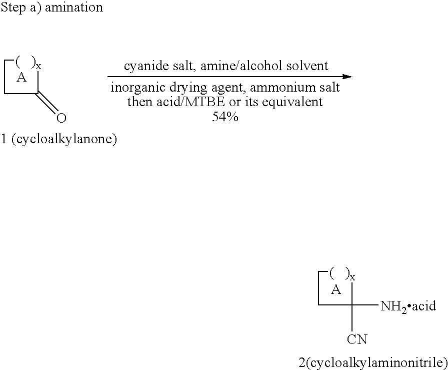
wherein:
A is an optionally partially or fully halogenated and optionally substituted with one or more OH, NH₂, C₁₋₆, SO₂, phenyl, CF₃;
X is C₀₋₈; 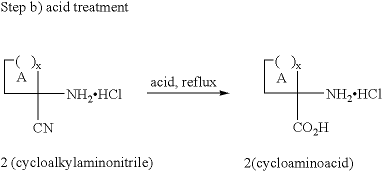
   wherein X is defined as immediately above.

In another embodiment of the invention X is 0 or 1.

In another embodiment of the invention methanol is used as the alcohol solvent.

In another embodiment of the invention the alcohol is removed before filtration of the inorganic salts.

The invention also provides for cycloaminonitrile compounds of general Formula II useful in the production of cycloalkylaminoacids as prepared using the methods described herein: 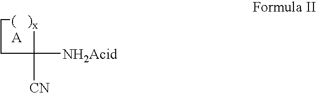
wherein A is a cycloalkyl optionally partially or fully halogenated and optionally substituted with one or more OH, NH₂, C₁₋₆, SO₂, phenyl, CF₃;
and X is 0 to 8.
   Terms and Definitions
   Chemical Nomenclature and Conventions Used

Terms not specifically defined herein should be given the meanings that would be given to them by one of skill in the art in light of the disclosure and the context. As used in the specification and appended claims, however, unless specified to the contrary, the following terms have the meaning indicated and the following conventions are adhered to.

The term 37 compounds of the invention” and equivalent expressions are meant to embrace the general formulas as herein described, including the tautomers, the prodrugs, the salts, particularly the pharmaceutically acceptable salts, and the solvates and hydrates thereof, where the context so permits. In general and preferably, the compounds of the invention and the formulas designating the compounds of the invention are understood to only include the stable compounds thereof and exclude unstable compounds, even if an unstable compound might be considered to be literally embraced by the compound formula. Similarly, reference to intermediates, whether or not they themselves are claimed, is meant to embrace their salts and solvates, where the context so permits. For the sake of clarity, particular instances when the context so permits are sometimes indicated in the text, but these instances are purely illustrative and it is not intended to exclude other instances when the context so permits.

The terms “optional” or “optionally” mean that the subsequently described event or circumstances may or may not occur, and that the description includes instances where the event or circumstance occurs and instances in which it does not. For example, “optionally substituted cycloalkyl” means that the cycloalkyl radical may or may not be substituted and that the description includes both substituted cycloalkyl radicals and cycloalkyl radicals having no substitution.

The term “substituted” means that any one or more hydrogens on an atom of a group or moiety, whether specifically designated or not, is replaced with a selection from the indicated group of substituents, provided that the atom's normal valency is not exceeded and that the substitution results in a stable compound. If a bond to a substituent is shown to cross the bond connecting two atoms in a ring, then such substituent may be bonded to any atom on the ring. When a substituent is listed without indicating the atom via which such substituent is bonded to the rest of the compound, then such substituent may be bonded via any atom in such substituent. Generally, when any substituent or group occurs more than one time in any constituent or compound, its definition on each occurrence is independent of its definition at every other occurrence. Such combinations of substituents and/or variables, however, are permissible only if such combinations result in stable compounds.

The yield of each of the reactions described herein is expressed as a percentage of the theoretical yield.

The term “pharmaceutically acceptable salt” means a salt of a compound of the invention which is, within the scope of sound medical judgment, suitable for use in contact with the tissues of humans and lower animals without undue toxicity, irritation, allergic response, and the like, commensurate with a reasonable benefit/risk ratio, generally water or oil-soluble or dispersible, and effective for their intended use. The term includes pharmaceutically-acceptable acid addition salts and pharmaceutically-acceptable base addition salts. As the compounds of the present invention are useful in both free base and salt form, in practice, the use of the salt form amounts to use of the base form. Lists of suitable salts are found in, e.g., S. M. Birge et al., J. Pharm. Sci., 1977, 66, pp. 1-19, which is hereby incorporated by reference in its entirety.

The term “hydrate” means a solvate wherein the solvent molecule(s) is/are H₂O.

The compounds of the present invention as discussed below include the free base or acid thereof, their salts, solvates, and prodrugs and may include oxidized sulfur atoms or quaternized nitrogen atoms in their structure, although not explicitly stated or shown, particularly the pharmaceutically acceptable forms thereof. Such forms, particularly the pharmaceutically acceptable forms, are intended to be embraced by the appended claims.

The term “isomers” means compounds having the same number and kind of atoms, and hence the same molecular weight, but differing with respect to the arrangement or configuration of the atoms in space. The term includes stereoisomers and geometric isomers.

The terms “stereoisomer” or “optical isomer” mean a stable isomer that has at least one chiral atom or restricted rotation giving rise to perpendicular dissymmetric planes (e.g., certain biphenyls, allenes, and spiro compounds) and can rotate plane-polarized light. Because asymmetric centers and other chemical structure exist in the compounds of the invention which may give rise to stereoisomerism, the invention contemplates stereoisomers and mixtures thereof. The compounds of the invention and their salts include asymmetric carbon atoms and may therefore exist as single stereoisomers, racemates, and as mixtures of enantiomers and diastereomers. Typically, such compounds will be prepared as a racemic mixture. If desired, however, such compounds can be prepared or isolated as pure stereoisomers, i.e., as individual enantiomers or diastereomers, or as stereoisomer-enriched mixtures. As discussed in more detail below, individual stereoisomers of compounds are prepared by synthesis from optically active starting materials containing the desired chiral centers or by preparation of mixtures of enantiomeric products followed by separation or resolution, such as conversion to a mixture of diastereomers followed by separation or recrystallization, chromatographic techniques, use of chiral resolving agents, or direct separation of the enantiomers on chiral chromatographic columns. Starting compounds of particular stereochemistry are either commercially available or are made by the methods described below and resolved by techniques well-known in the art.

The term “enantiomers” means a pair of stereoisomers that are non-superimposable mirror images of each other.

The terms “diastereoisomers” or “diastereomers” mean optical isomers which are not mirror images of each other.

The terms “racemic mixture” or “racemate” mean a mixture containing equal parts of individual enantiomers.

The term “non-racemic mixture” means a mixture containing unequal parts of individual enantiomers.

Some of the compounds of the invention can exist in more than one tautomeric form. As mentioned above, the compounds of the invention include all such tautomers.

It is well-known in the art that the biological and pharmacological activity of a compound is sensitive to the stereochemistry of the compound. Thus, for example, enantiomers often exhibit strikingly different biological activity including differences in pharmacokinetic properties, including metabolism, protein binding, and the like, and pharmacological properties, including the type of activity displayed, the degree of activity, toxicity, and the like. Thus, one skilled in the art will appreciate that one enantiomer may be more active or may exhibit beneficial effects when enriched relative to the other enantiomer or when separated from the other enantiomer. Additionally, one skilled in the art would know how to separate, enrich, or selectively prepare the enantiomers of the compounds of the invention from this disclosure and the knowledge of the prior art.

Thus, although the racemic form of drug may be used, it is often less effective than administering an equal amount of enantiomerically pure drug; indeed, in some cases, one enantiomer may be pharmacologically inactive and would merely serve as a simple diluent. For example, although ibuprofen had been previously administered as a racemate, it has been shown that only the S-isomer of ibuprofen is effective as an anti-inflammatory agent (in the case of ibuprofen, however, although the R-isomer is inactive, it is converted in vivo to the S-isomer, thus, the rapidity of action of the racemic form of the drug is less than that of the pure S-isomer). Furthermore, the pharmacological activities of enantiomers may have distinct biological activity. For example, S-penicillamine is a therapeutic agent for chronic arthritis, while R-penicillamine is toxic. Indeed, some purified enantiomers have advantages over the racemates, as it has been reported that purified individual isomers have faster transdermal penetration rates compared to the racemic mixture. See U.S. Pat. Nos. 5,114,946 and 4,818,541.

Thus, if one enantiomer is pharmacologically more active, less toxic, or has a preferred disposition in the body than the other enantiomer, it would be therapeutically more beneficial to administer that enantiomer preferentially. In this way, the patient undergoing treatment would be exposed to a lower total dose of the drug and to a lower dose of an enantiomer that is possibly toxic or an inhibitor of the other enantiomer.

Preparation of pure enantiomers or mixtures of desired enantiomeric excess (ee) or enantiomeric purity are accomplished by one or more of the many methods of (a) separation or resolution of enantiomers, or (b) enantioselective synthesis known to those of skill in the art, or a combination thereof. These resolution methods generally rely on chiral recognition and include, for example, chromatography using chiral stationary phases, enantioselective host-guest complexation, resolution or synthesis using chiral auxiliaries, enantioselective synthesis, enzymatic and nonenzymatic kinetic resolution, or spontaneous enantioselective crystallization. Such methods are disclosed generally in Chiral Separation Techniques: A Practical Approach (2nd Ed.), G. Subramanian (ed.), Wiley-VCH, 2000; T. E. Beesley and R. P. W. Scott, Chiral Chromatography, John Wiley & Sons, 1999; and Satinder Ahuja, Chiral Separations by Chromatography, Am. Chem. Soc., 2000. Furthermnore, there are equally well-known methods for the quantitation of enantiomeric excess or purity, for example, GC, HPLC, CE, or NMR, and assignment of absolute configuration and conformation, for example, CD ORD, X-ray crystallography, or NMR.

In general, all tautomeric forms and isomeric forms and mixtures, whether individual geometric isomers or stereoisomers or racemic or non-racemic mixtures, of a chemical structure or compound is intended, unless the specific stereochemistry or isomeric form is specifically indicated in the compound name or structure.

Cycloalkyanones—It is understood that different cycloalkanones such as cyclobutanone can be used in the invention. Cycloalkanones can be prepared according to the general process described in Cycloalkanones are classically prepared by the Dieckmann condensation (Schaefer, J. P., and Bloomfield, J. J. Org. React. 1967, 15, 1-203), yet they can also be prepared by oxidation of the appropriate alcohol. Cycloalkanones are also commercially available. The preferred cycloalkylalanone is cyclobutanone.

Solvents—It is understood that a number of different solvents can be used in the present invention. Acceptable solvents include linear and branched alcohols containing 1-5 carbons but are not limited to the list consisting of Methanol, ethanol, propanol, butanol and isopropanol, sec-butanol, tert-butanol. The anhydrous alcohol helps prevent premature hydrolysis of the nitrile and accelerate the formation of the aminonitrile. The preferred solvent is methanol.

Cyanide salts—It is understood that different cyanide salts can be used in the present invention. Acceptable cyanide salts include but are not limited to the list consisting of, NaCN, KCN, LiCN, TMSCN. The preferred cyanide salt is NaCN.

Amines—It is understood that agents other than NH₃ that could be converted into a subsequent step to a primary amine could also be utilized in the present invention. Aliphatic primary amines may be used. The preferred agent is NH₃.

Inorganic drying agent—An inorganic drying agent may be used in the invention. Suitable inorganic drying agents can include but are not limited to MgSO₄, NaSO₄ and molecular sieves. The preferred drying agent is MgSO₄.

Hydrolyzing agents—It is understood that a number of hydrolyzing agents can be used in the invention. Hydrolyzing agents are preferably aqueous agents for example phosphoric, sulfuric, sulfonic, trifluoroacetic, trifluoromethansulfonic and hydrochloric acids. The most preferred hydrolyzing agent is hydrochloric acid.

Buffered Solution—It is understood that a buffered solution can be used in the invention and that by having a base such as NH₃ and a weak acid (NH₄Cl) present that better conversion can be achieved. Other bases and weak acids that can be used include NH₄OAc, NH₄NO₃ and (NH₄₎₂SO₄.

General Synthetic Methods

The present invention provides for compositions of cycloalkylaminoacids of general Formula I and to processes for preparing the same. 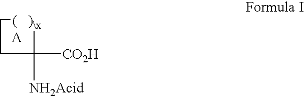
wherein X, and A are as defined herein.

The invention also provides processes for making compounds of Formula (I). Intermediates used in the preparation of compounds of the invention are either commercially available or readily prepared by methods known to those skilled in the art.

Optimum reaction conditions and reaction times may vary depending on the particular reactants used. Unless otherwise specified, solvents, temperatures, pressures, and other reaction conditions may be readily selected by one of ordinary skill in the art. Specific procedures are provided in the synthetic examples section. Typically, reaction progress may be monitored by HPLC or thin layer chromatography (TLC), if desired, and intermediates and products may be purified by chromatography on silica gel and/or by recrystallization. 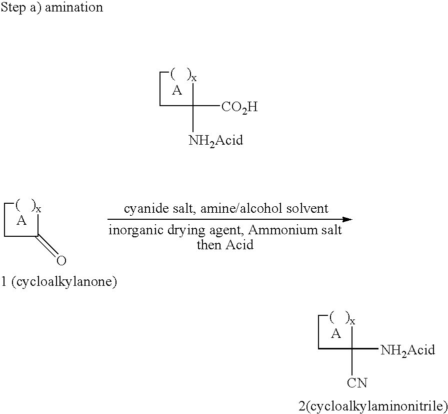
wherein:
A is a cycloalkyl optionally partially or fully halogenated and optionally substituted with one or more OH, NH₂, C_{1-6,} SO₂, phenyl, CF₃;
X is C₀₋₈.
   Procedure

A flask, reactor, or otherwise suitable container is assembled for reflux condensation with mechanical agitation under an inert atmosphere. The container is evacuated and inerted, then charged with 2-100 equivalents of an inorganic drying agent such as MgSO₄, Na₂SO₄, or molecular sieves and cyanide salt. An ammonium salt such as NH₄Cl or NH₄OAc is then added, using 0.1 to 10 molar equivalents relative to the ketone used. The vessel is then inerted again, and charged with a solution of NH₃ in an anhydrous alcohol. Linear and branched alcohols containing 1-5 carbons may be used, and the NH₃ concentration may range from saturated (dependent on the alcohol used, often 4-5 M) to dilute, ˜0.25M. The NH₃ molar equivalents must exceed the molar equivalents of the ketone used. To this well agitated mixture is then added the ketone, either neat or as a solution in an appropriate alcohol. The mixture is then stirred for 1 to 48 hours at 0° C. to ˜60° C., preferably from 25° C. to ˜60° C., until analysis reveals consumption of the ketone. The mixture is cooled and the solvents removed under vacuum at ambient temperature. Low or high vacuum may be used, and any non-polar aprotic organic solvent may be added at any time to azeotropically remove the alcohol. Preferred aprotic agents include EtOAc, iPrOAc, Et₂O, MTBE, di-butyl ether, heptane, cyclohexane, methylcyclohexane and toluene. When analysis reveals the alcohol content is less than 5% by volume, the resultant slurry is cooled to 0° C. to 40° C. and filtered or centrifuged under an inert atmosphere to remove all inorganic impurities. The filtrate containing the aminonitrile is then treated with an anhydrous acid solution to precipitate the aminonitrile acid salt.

Removal of the polar alcohol solvent is done before filtration of the inorganic salts. Since the inorganic salts have some solubility in the alcohol solvent, performing the filtration first would ensure that the product will be contaminated with inorganic impurities. Performing the filtration after removal of the alcohol therefore leads to product which is free of inorganic impurities. This is considered advantageous, because the final product, the aminoacid, will be soluble in all the same solvents that the inorganics are soluble in, rendering purification very difficult.

The acid used may be any of the organic or inorganic acids dissolved in a non-polar organic solvent, or added as a gas. The acid concentration may range from 0.1M to 6M, and the equivalents of acid should be at least 75% of the ketone charge on a molar basis. The resultant slurry is then agitated from 0.1 to 48 hours at any temperature between ˜80° C. to 25° C. to complete formation of the salt. The resultant slurry is then filtered or centrifuged under an inert atmosphere to isolate the aminonitrile acid salt as a solid. This salt may then be dried to constant weight, or optionally washed with 5-500% by volume of the original batch volume, and then dried to constant weight. The filtrate may be held at reduced temperature and later refiltered or centrifuged to obtain a second crop of aminonitrile acid salt.

Is also considered advantageous for the conversion of the aminonitrile to its acid salt to occur in an organic solvent. This allows for removal of any organic impurities which may be present. Through the combination of inorganic impurity removal, and organic impurity removal here, the aminonitrile acid salt is generated in very high purity. This in turn leads to generation of the aminoacid in the hydrolysis step in very high yield and purity. High purity is considered 90% and most preferably 95%. 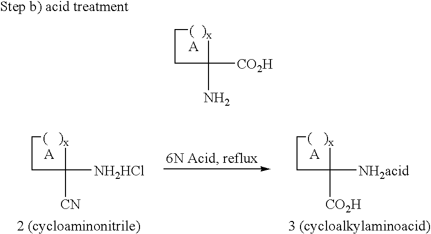
wherein A and X are defined as immediately above.
Procedure

The aminonitrile acid salt is charged to a flask, reactor, or other suitable vessel. An aqueous solution of a strong acid is then added. A polar cosolvent such as C₁₋₅ alcohol, or glymes may optionally be added. The choice of acids is broad, including HCl, H₂SO₄, HNO₃, H₃PO₄, methanesulfonic acid, and other strong inorganic and organic acids. The concentration of acid may range from 2M to 20M. The hydrolysis is then carried out until analysis indicates the nitrile has been hydrolyzed. This would occur between 25° C. and the boiling point of the solvent. At the conclusion of the reaction, the solvents are removed in vacuo to give the aminoacid product as it's acid salt. Polar solvents may be added to azeotropically dry the product solution. If the zwitterion is desired, the pH is adjusted with any suitable base to near the isoelectronic point of the aminoacid, and the product isolated as a solid precipitate, or following extraction of the aqueous mixture with any suitable organic solvent.

### SYNTHETIC EXAMPLES

In order for this invention to be more fully understood, the following examples are set forth. These examples are for the purpose of illustrating embodiments of this invention, and are not to be construed as limiting the scope of the invention in any way since, as recognized by one skilled in the art, particular reagents or conditions could be modified as needed for individual compounds.

### EXAMPLE 1

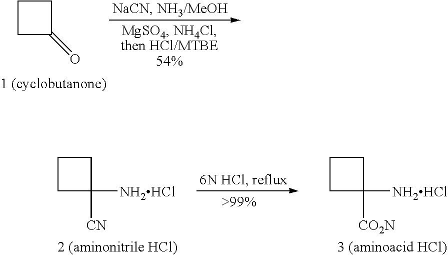

Aminonitrile HCl 2. A 4-neck IL round bottom flask with mechanical stirrer and reflux condenser was evacuated/N₂ filled (3 times), then charged with 23.6 g MgSO₄ (excess), 6.71 g NaCN (137 mmol, 1.02 eq.), and 3.53 g NH₄Cl (67.4 mmol, 0.5 eq.). The flask was again evacuated/N₂ filled (3 times), then 168 mL 4.9M NH₃/MeOH (825 mmol, 6.1 eq.) was added. The stirrer was started, then 10.0 mL cyclobutanone 1 (134 mmol, 1 eq.) was added neat. The mixture was then stirred 16 hours at ambient temperature under N₂, then heated at 55° C. for 5 hours. The mixture was cooled and all sovents removed under high vacuum at ambient temperature. The residue was then suspended in 300 mL MTBE and filtered under N₂ into a round bottom flask, using 150 mL MTBE to wash the solids. The filtrate was then immediately cooled to 0° C. and treated dropwise with 75 mL 2.87M HCl/MTBE (215 mmol, 1.6 eq.). After stirring 2 hours at 0° C., the slurry was filtered under N₂ and the solid collected. The filtrate was cooled to 0° C. and refiltered. All solids were washed with 150 mL MTBE under N₂ to give 9.5 g aminonitrile HCl 2 (54%) as a colorless solid. ¹³C NMR (below) showed a pure compound. ¹³C NMR (100 MHz, DMSO)δ: 119.20 (s), 46.29 (s), 31.44 (t), 14.66 (t).

Aminoacid HCl 3. 1.00 g aminonitrile HCl (7.55 mmol, 1 eq.) was dissolved in 10 mL 6N HCl and heated to reflux under N₂. After 12 hours, the mixture was cooled to ambient temperature and the volatiles removed under high vacuum, azeotroping with methanol to remove the last traces of H₂O, giving 1.15 g aminoacid HCl 3 (>99%) as a colorless solid. ¹³C NMR (below) showed a pure compound. ¹³C NMR (100 MHz, DMSO)δ: 172.41 (s), 56.37 (s), 29.30 (t), 14.48 (t). The structure was confirmed without question by converting a sample of commercial aminoacid (Narchem Lot 45-34-D) to its HCl salt with 6N HCl, and obtaining ¹³C NMR spectra. It showed identical ¹³C NMR resonances to the synthetic sample described above.

1. Cycloalkylaminoacid compounds of Formula I: 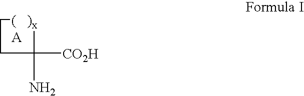
wherein
A is a cycloalkyl optionally partially or fully halogenated and optionally substituted with one or more OH, NH₂, C₁₋₆, SO₂, phenyl or CF₃;
X is C₀₋₈
and pharmaceutically acceptable salts, salts, solvates, hydrates, stereoisomers, optical isomers; enatiomers, diastereoisomes and racemeic mixtures, esters, tautomers, individual isomers, and mixtures of isomers thereof.

2. The compound of claim 1 wherein X is 0 or 1.

3. A process for preparing cycloalkylaminoacids of Formula I 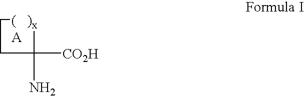
wherein A is a cycloalkyl optionally partially or fully halogenated and optionally substituted with one or more OH, NH₂, C₁₋₆, SO₂, phenyl or CF₃;
X is C₀₋₈
comprised of the steps of:
a) performing an amination of a cycloalanone with a cyanide salt, an amine and an alcohol solvent to provide a cycloalkylaminonitrile;
b) treatment of the product of step A with an acid to provide a cycloaminoacid.

4. The process of claim 3 wherein the salt is chosen from NaCN, KCN, LiCN, or TMSCN.

5. The process of claim 3 wherein the salt is NaCN.

6. The process of claim 3 wherein the alcohol is chosen from methanol, ethanol, propanol, butanol and isopropanol, sec-butanol or tert-butanol.

7. The process of claim 3 wherein the alcohol is methanol

8. The process of claim 3 wherein the alcohol is removed before filtration of the inorganic salts.

9. The process of claim 3 for making compounds of Formula I: 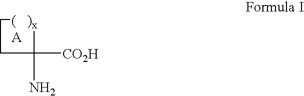
wherein
A is a cycloalkyl optionally partially or fully halogenated and optionally substituted with one or more OH, NH₂, C₁₋₆, SO_{2,} phenyl or CF₃;
X is C₀;
Comprised of the steps of:
a) performing an amination of a cycloalanone with a sodium cyanide salt, an amine and methanol to provide a cyclobutylaminonitrile;
b) treatment of the product of step A with HCl to provide a cycloaminoacid.

10. Compounds of general Formula II: 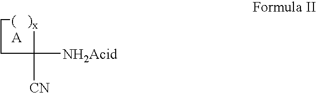
wherein A is a cycloalkyl optionally partially or fully halogenated and optionally substituted with one or more OH, NH₂, C₁₋₆, SO₂, phenyl, CF₃;
and X is 0 to 8.
